(19) United States
(12) Patent Application Publication
(10) Pub. No.: US 2009/0082872 A1
(43) Pub. Date: Mar. 26, 2009

(54) Intervertebral implant

(75) Inventor: Jens Beger, Tuttlingen (DE)

Correspondence Address: Lipsitz & McAllister, LLC, 755 MAIN STREET, MONROE, CT, 06468, US

(73) Assignee: Aesculap AG, Tuttlingen (DE)

(21) Appl. No.: 12/284,330

(22) Filed: Sep. 19, 2008

Related U.S. Application Data
(63) Continuation of application No. PCT/EP2007/002675, filed on Mar. 27, 2007.

(30) Foreign Application Priority Data
Apr. 6, 2006 (DE) 10 2006 016 985

(51) Int. Cl. A61F 2/44

(52) U.S. Cl. 623/17.16; 623/17.11

(57) ABSTRACT

In an intervertebral implant with at least one abutment surface formed from a plurality of parts for a vertebral body, which parts are movable relative to one another between an insertion position, in which the intervertebral implant has a small cross section, and a working position, in which the intervertebral implant has a larger cross section and in which the parts jointly form the abutment surface, and with an adjusting means for moving the parts out of the insertion position into the working position, in order to provide an uncomplicated structure having small dimensions, it is proposed that the adjusting means have at least one expansion body, which increases in volume when filled with a flowable medium and thus moves the parts into the working position.

This application is a continuation of international application number PCT/EP2007/002675 filed on Mar. 27, 2007.

The present disclosure relates to the subject matter disclosed in international application PCT/EP2007/002675 of Mar. 27, 2007 and German application number 10 2006 016 985.9 of Apr. 6, 2006, which are incorporated herein by reference in their entirety and for all purposes.

### BACKGROUND OF THE INVENTION

The invention relates to an intervertebral implant with at least one abutment surface formed from a plurality of parts for a vertebral body, said parts being movable relative to one another between an insertion position, in which the intervertebral implant has a small cross-section, and a working position, in which the intervertebral implant has a larger cross-section and in which the parts jointly form the abutment surface, and with an adjusting means for moving the parts out of the insertion position into the working position.

In the case of intervertebral implants, which serve to replace intervertebral discs, for example, it is important that the intervertebral implant abuts against the adjacent vertebral bodies with as large an abutment surface as possible in order to keep the compressive forces that arise as low as possible and thus prevent the intervertebral implant from collapsing or fusing into the vertebral body. The size of the abutment surface should preferably be selected so that it is equal to or only slightly smaller than the face of the abutting vertebral body.

However, the relatively large configuration of the abutment surface causes difficulties during insertion of the implant into the body, in particular during insertion through a dorsal or dorsal-lateral access point.

It is therefore known to form the abutment surface in intervertebral implants from a plurality of parts, which are movable relative to one another and are arranged in an insertion position so that the cross-section of the intervertebral implant is relatively small, and therefore in this insertion position of the parts the implant can also be inserted into the body through a small access point of the body. Inside the body the parts are then moved into a working position, in which they open out the final and larger abutment surface. The implant is thus provided with a larger cross-section overall, but this is not problematic, since this larger cross-section is only assumed after the implant is positioned inside the body.

However, movement of the parts out of the insertion position into the working position inside the body is difficult. This movement must be performed, for example, by instruments inserted into the body, and this is difficult, and in this case injuries can also occur because of the forces employed during this.

It is also known to provide an adjusting means, which in the known case operates with a spindle drive (WO 2004/103226), to increase the size of the abutment surface on the implant in the case of an intervertebral implant with movable parts. However, the structure of the implant is complicated as a result of this and this also leads to increased dimensions.

It is an object of the invention to propose a possibility for moving the parts of the abutment surface, in which case the structure of the implant remains relatively simple and the dimensions of the implant are increased as little as possible.

### SUMMARY OF THE INVENTION

This object is achieved according to the invention with an intervertebral implant of the above-described type in that the adjusting means has at least one expansion body, which increases in volume when filled with a flowable medium and thus moves the parts into the working position.

Such an expansion body, which can be in the form of a bellows or a balloon, for example, can be arranged in the implant in a simple manner, and when in unfilled state such an expansion body assumes a minimum volume, so that the structural size of the implant can remain practically unchanged. By filling the expansion body with a flowable medium, e.g. a gas or a liquid, in particular with a sodium chloride solution, the volume of the expansion body can be increased, and because of its increased volume this enlarged expansion body moves the parts of the abutment surface out of the insertion position into the working position. In this case, different kinematic arrangements are possible, e.g. the parts can be displaceable parallel to one another, the expansion body then displaces the parts accordingly when increasing in volume.

In another exemplary embodiment parts can be pivoted against one another by the expansion body.

After the movement of the parts into the working position, the parts must be secured in this position, so that they can perform their support function in the entire region of the abutment surface.

In a particularly preferred embodiment of the invention, it is provided that besides the expansion body it has a core composed of a swellable material, which has a low volume in a state low in liquid and upon liquid absorption increases its volume, and that after liquid absorption with increased volume the core supports the parts of the abutment surface in the working position in such a manner that they are prevented from moving back into the insertion position.

After insertion of the intervertebral implant into the body, the swellable material comes into contact with body fluid and starts to increase in volume. This can take a few hours. In this period the parts of the implant are held in the working position by the expansion body. As the volume of the core increases, this support function is taken over by the core, and the expansion body can then be emptied and/or removed.

The swellable material of the core not only serves to support the parts in the working position, but also acts as a cushion between the end faces of the intervertebral implant. The swellable material thus assumes a function between adjacent vertebral bodies that corresponds to the function of the removed intervertebral disc, which likewise acts as a cushion and joint, and supports the vertebral bodies so that they are displaceable and can pivot to a small degree relative to one another, while also acting as a shock absorber between both vertebral discs.

The swellable material can be a hydrogel, for example.

In a preferred embodiment, it is provided that the expansion body has at least one outlet for the flowable material, which is arranged in such a manner that the flowable medium discharging from it impinges against the swellable low-liquid material of the core. In this way, as a result of the flowable medium discharging from the outlet, the flowable medium, in particular a sodium chloride solution, can act on the swellable material of the core. This accelerates the absorption of liquid of the swellable material and thus its increase in volume.

It is favourable if the core is arranged between two expansion bodies.

In a preferred embodiment, the abutment surface has a central abutment section and a respective lateral abutment section mounted to pivot thereon on opposite sides, and each lateral section has a respective associated expansion body. As a result of this, a very compact structure is obtained, in which the abutment surface can be enlarged by folding out the lateral abutment sections in a similar manner to a table with folding ends. The folding out occurs as a result of a respective expansion body associated with each lateral abutment section.

In particular, the expansion bodies and when applicable a core of swellable material can be fixed to the central abutment section.

It is possible, in principle, that an intervertebral implant only has such an abutment surface on one side, but the intervertebral implant is generally configured so that an abutment surface comprising a plurality of parts is respectively provided on opposite sides of the intervertebral implant, and that the expansion body or bodies and when applicable the core of swellable material are arranged in the space between the two abutment surfaces. This results in a particularly compact arrangement of the intervertebral implant, in particular in the insertion position of the parts of the abutment surface.

The expansion bodies can be detachably held on the intervertebral implant, so that they can be removed by other means after the parts move into the working position and these parts are supported in the working position.

It is also possible that the expansion bodies are made of an resorbable material, so that after their movement task has finished and after emptying, they firstly remain in the body and are then gradually broken down by the body.

Polyglycolic acid, poly-p-dioxanone, copolymers of glycolic acid and/or trimethyl carbonate and/or caprolactone and/or p-dioxanone and/or lactic acid, for example, can be used as resorbable material. These substances can be used in different proportions by weight and in a wide variety of combinations.

The following description of preferred embodiments of the invention serves as a more detailed explanation in association with the drawing.

### BRIEF DESCRIPTION OF THE DRAWINGS

FIG. 1 is a perspective view of a vertebral body and an intervertebral implant arranged thereon with the parts of the abutment surfaces in the insertion position;

FIG. 2 is a perspective view of the intervertebral implant of FIG. 1 between two vertebral bodies with the parts of the abutment surface in the working position and with filled expansion bodies;

FIG. 3 is view similar to FIG. 2 with a core of swellable material after liquid absorption and increase in volume;

FIG. 4 is a perspective view of the intervertebral implant of FIG. 1 with an abutment surface lifted off, with the parts of the abutment surface in the working position and with the core and the expansion bodies before their increase in volume by liquid absorption or filling with a flowable medium;

FIG. 5 is a view in partial section taken along line 5-5 in FIG. 6 in the mounting region of a lateral abutment plate against a central abutment plate with a locking device, and

FIG. 6 is a plan view onto the pivot mounting region shown in FIG. 5.

### DETAILED DESCRIPTION OF THE INVENTION

The intervertebral implant 1 shown in the drawing is used as a replacement for a removed intervertebral disc between two adjacent vertebral bodies 2, 3 and is respectively supported against the faces of these two vertebral bodies 2, 3 facing towards one another.

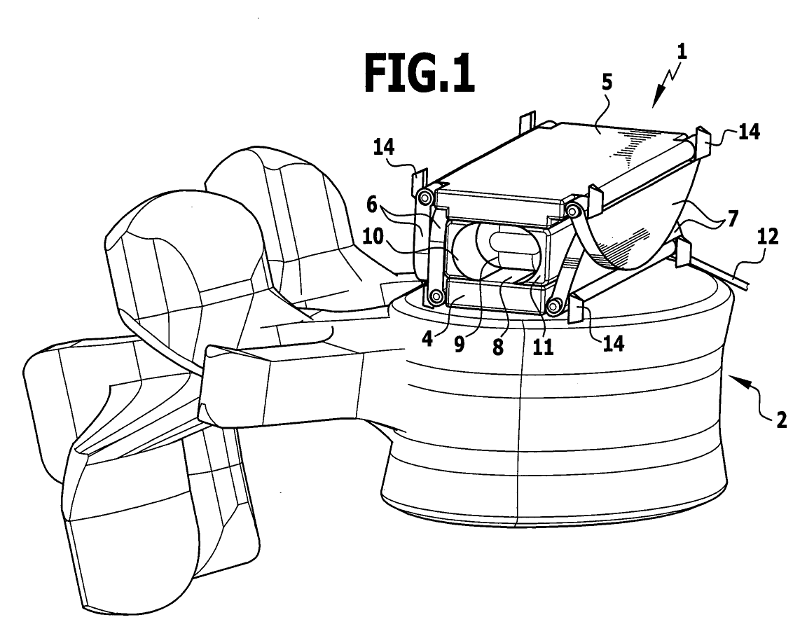

For this, the intervertebral implant has two spaced central abutment plates 4, 5 running parallel to one another, which have a substantially rectangular cross-section and on their opposite longitudinal sides two lateral abutment plates 6 and 7 are respectively disposed to pivot around an axis running parallel to the longitudinal sides. A respective central abutment plate 4 or 5 and the abutment plates 6 and 7 disposed thereon jointly form an end plate of the intervertebral implant 1. In this case, the two end plates are substantially the same in configuration and only differ in that one end plate is slightly wider than the other, so that the lateral abutment plates 6, 7 of the two end plates lie one on top of the other when they project perpendicularly from the central abutment plates 4, 5 (FIG. 1). This arrangement, in which the lateral abutment plates 6, 7 lie one on top of the other running perpendicularly to the central abutment plates 4, 5, will hereafter be referred to as the insertion position. In this position of the abutment plates 6, 7 a particularly small cross-sectional area of the intervertebral implant results, so that it is also possible to insert the implant in this insertion position through an opening in the body when small insertion openings are used.

Located in the centre in the space 8 between the abutment plates 4 and 5 is a core 9 of swellable material, which extends substantially over the length of the central abutment plates 4, 5 and is arranged between two expansion bodies 10, 11, which extend substantially over the length of the central abutment plates 4, 5 and extend on both sides of the core 9 along the longitudinal sides of the central abutment plates 4, 5. The core is made of a swellable material, which has a small volume after the extraction of liquid, in particular water, but can readily absorb surrounding liquid and in so doing increases its volume. The increase in volume can be anisotropic, i.e. can preferably occur in specific directions.

In principle, all non-degradable hydrophilic polymers are conceivable as materials in this case. Examples are polyacrylic acid and its derivatives such as polymethacrylic acid, polyacrylamide, polyacrylonitrile, polyacrylate, polyhydroxy ethyl methacrylates, or other substances such as e.g. polyvinylpyrrolidone (PVP), polyurethanes, high-molecular polyvinyl alcohol.

Also conceivable are polymer blends (copolymers linked to one another by means of bonds) composed of the abovementioned polymers or interpenetrating networks (IPN) composed of the abovementioned polymers. IPN consist of at least two different polymers, the polymer chains of which are entangled and are linked to one another by means of physical interactions (van der Waals, electrostatic, H-bridge bonds and/or ionic forces).

Further polymer mixtures that can be used are copolymers and also IPN of polyacrylates (polyacrylic acid and its derivatives such as polymethacrylic acid, polyacrylamide, polyacrylonitrile, polyacrylate) with polycaprolactone.

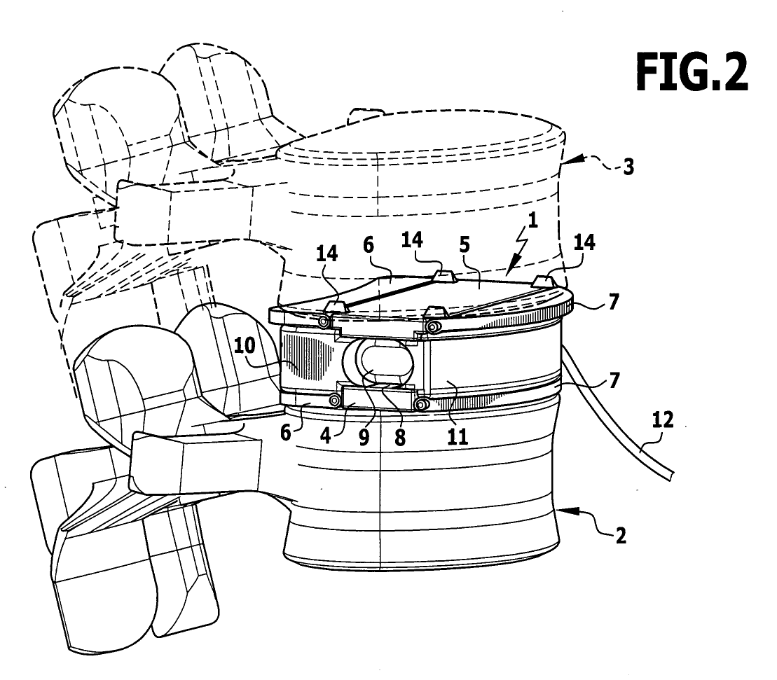

In the illustrated exemplary embodiment, the expansion bodies 10, 11 are configured as balloon-like cavities, which abut closely against the core 9 and have pliant, and if necessary also extensible, walls. The two expansion bodies 10, 11 are connected to feed tubes 12, 13, through which a flowable medium can be fed into the expansion bodies 10, 11. This can be a gas, but is preferably a liquid, in particular a sodium chloride solution. As a result of filling the expansion bodies 10, 11 with flowable medium, the volume of the expansion bodies 10, 11 is increased, either by the walls of the expansion bodies 10, 11 folding out or by elastic extension of the walls of the expansion bodies 10, 11, or by a combination of the two effects. This increase in volume of the expansion bodies 10, 11 causes the expansion bodies 10, 11 to expand laterally away from the core 9 against the folded-down lateral abutment plates 6, 7 and these are thus respectively pivoted outwards until the lateral abutment plates 6, 7 are pivoted about 90° outwards and extend in a plane with the central abutment plates 4, 5, on which they are hinged (FIG. 2). They then form together with the central abutment plates 4 and 5 an abutment surface, which is substantially larger than the area of the central abutment plates 4, 5 and also the cross-section of the intervertebral implant 1 is then significantly larger than when the lateral abutment plates 6, 7 are positioned in the insertion position (FIG. 1).

In the filled state, the expansion bodies 10, 11 thus extend over the space 8 between the central abutment plates 4, 5 laterally into the space between the folded-out lateral abutment plates 6, 7 and thus fill the entire space between the end plates of the intervertebral implant 1 except for the core 9.

The folding out movement of the lateral abutment plates 6, 7 is restricted by stops 14, which are arranged on the lateral abutment plates 6, 7 and abut against the central abutment plates 4, 5, as soon as the lateral abutment plates 6, 7 are coplanar with the central abutment plates 4, 5.

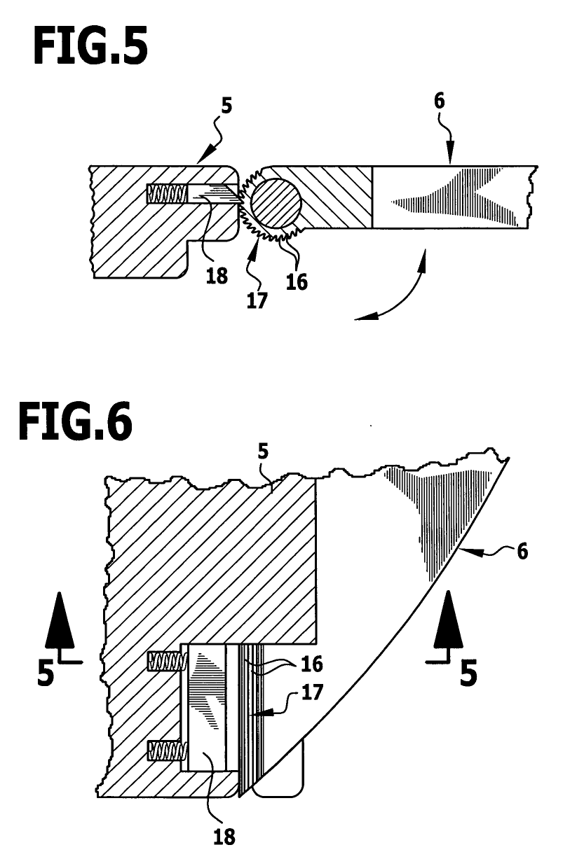

After the lateral abutment plates 6, 7 have been folded out into the position referred to as the working position, in which they form an enlarged abutment surface together with the central abutment plates 4, 5, the lateral abutment plates 6, 7 must be fixed in the folded-out position. For this, in the exemplary embodiment of FIGS. 5 and 6, in the region of the pivot mounting the lateral abutment plates 7 are fitted on the outside with a locking face 17 provided with detents 16, and there are mounted in the central abutment plate 5 spring-loaded locking elements 18, which are displaceable transversely to the pivot axis, abut against the locking face 17 and slide along the locking face 17 when the lateral abutment plate 7 pivots. In this case, the geometry of the detents 16 and the locking elements 18 is selected so that the parts can slide along one another in one direction, while in the opposite direction locking occurs by engagement of the locking element 18 into the detents 16 and the lateral abutment plate 7 is thus locked in position. In other words, each lateral abutment plate 7 can only be pivoted out of the insertion position into the implant position, and not in the reverse direction.

The same arrangement can naturally also be used with the other lateral abutment plate 6.

Instead of fixing the lateral abutment plates 6, 7 by mechanical locking means, or in addition to this fixture by the mechanical locking means, it can be additionally provided that a fixture of the lateral abutment plates 6, 7 occurs by means of the core 9.

This core 9 is inserted in a low-liquid state, i.e. with a low volume, during insertion of the implant.

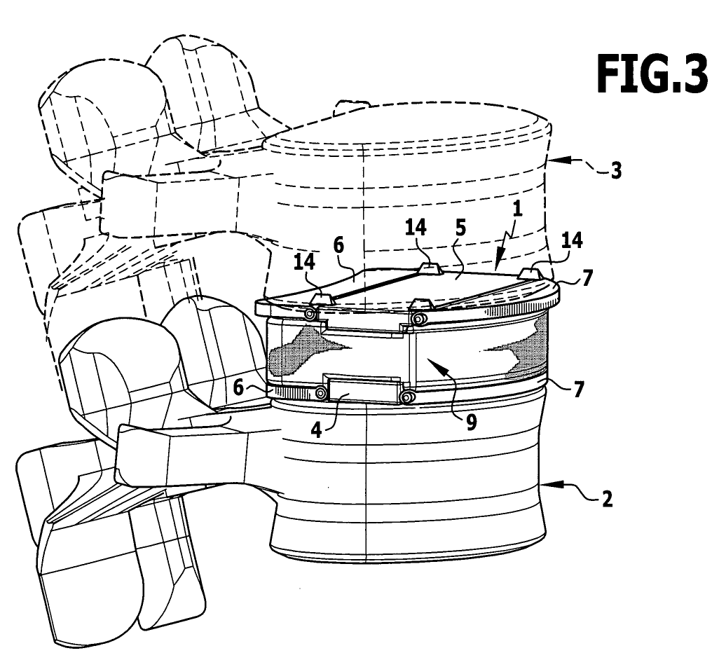

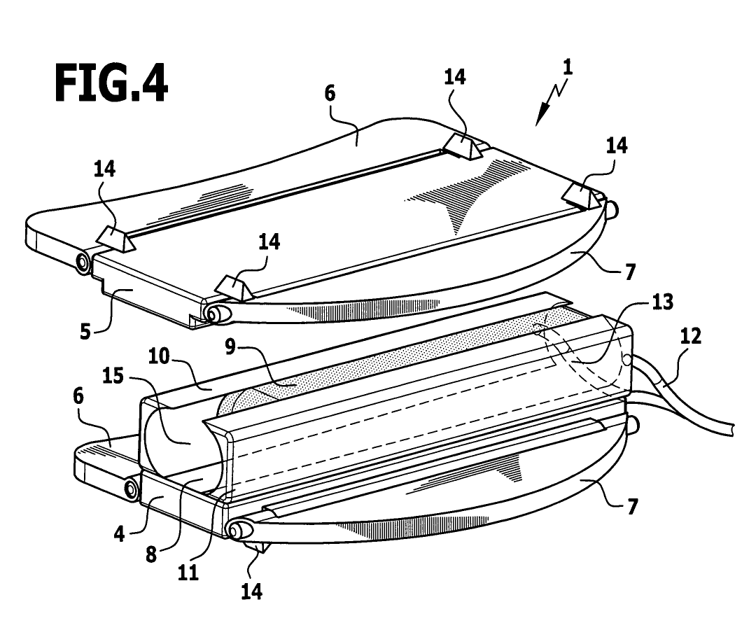

After insertion into the body, the material of the core 9 comes into contact with surrounding liquid, possibly also with liquid discharging from the expansion bodies 10, 11 through outlets 15. The contact with the liquid leads to a liquid absorption through the core 9 and thus to a significant increase in volume of the core 9, which as a result of this increase in volume expands into the entire space between the central abutment plates 4, 5 and between the folded-out lateral abutment plates 6, 7 (FIG. 3). The expansion bodies 10 can be emptied in this state, so that they do not inhibit the expansion of the core 9, or they are removed completely by the operating surgeon, since they have fulfilled their purpose as a means for pivoting out the lateral abutment plates 6, 7.

After expansion of the core 9, this forms a cushion between the two folded-out end plates, which allows some mobility of the end plates relative to one another and at the same time acts as a shock absorber between the adjacent vertebral bodies 2, 3.

Because of the combination of the swellable core 9 and the fillable expansion bodies 10, 11, it is possible, in spite of the time-consuming expansion process of the core 9 that can extend over hours, to move the lateral abutment plates 6, 7 of the intervertebral implant 1 very quickly into the working position during an operation and then, after conclusion of the expansion process of the core 9, leave the function of supporting the end plates completely to said core without the expansion bodies 10, 11 having to also take part in this supporting function.

1. Intervertebral implant with at least one abutment surface formed from a plurality of parts for a vertebral body, said parts being movable relative to one another between an insertion position, in which the intervertebral implant has a small cross-section, and a working position, in which the intervertebral implant has a larger cross-section and in which the parts jointly form the abutment surface, and with an adjusting means for moving the parts out of the insertion position into the working position, wherein the adjusting means has at least one expansion body, which increases in volume when filled with a flowable medium and thus moves the parts into the working position.

2. Implant according to claim 1, wherein the expansion body is a bellows.

3. Implant according to claim 1, wherein the expansion body is a balloon.

4. Implant according to claim 1, wherein the flowable medium is a gas.

5. Implant according to claim 1, wherein the flowable medium is a liquid.

6. Implant according to claim 5, wherein the flowable medium is a sodium chloride solution.

7. Implant according to claim 1, wherein besides the expansion body it has a core composed of a swellable material, which has a small volume in a state low in liquid and upon liquid absorption increases its volume, and after liquid absorption with increased volume the core supports the parts of the abutment surface in the working position in such a manner that they are prevented from moving back into the insertion position.

8. Implant according to claim 7, wherein the swellable material is a hydrogel.

9. Implant according to claim 7, wherein the expansion body has at least one outlet for the flowable material, which is arranged in such a manner that the flowable medium discharging from it impinges against the swellable low-liquid material of the core.

10. Implant according to claim 7, wherein the core is arranged between two expansion bodies.

11. Implant according to claim 1, wherein the abutment surface has a central abutment section and a respective lateral abutment section mounted to pivot thereon on opposite sides, and each lateral section has a respective associated expansion body.

12. Implant according to claim 11, wherein the expansion bodies and when applicable a core of swellable material are fixed to the central abutment section.

13. Implant according to claim 1, wherein an abutment surface comprising a plurality of parts is respectively provided on opposite sides of the intervertebral implant, and the expansion body or bodies and when applicable the core of swellable material are arranged in the space between the two abutment surfaces.

14. Implant according to claim 1, wherein the expansion bodies are detachably held on the intervertebral implant.

15. Implant according to claim 1, wherein the expansion bodies are made of an resorbable material.

16. Implant according to claim 9, wherein the core is arranged between two expansion bodies.
